(19) United States
(12) Patent Application Publication
(10) Pub. No.: US 2003/0103984 A1
(43) Pub. Date: Jun. 5, 2003

(54) Fusion proteins of biologically active peptides and antibodies

(76) Inventor: Heinz Kohler, Lexington, KY (US)

Correspondence Address: ANTONELLI TERRY STOUT AND KRAUS, SUITE 1800, 1300 NORTH SEVENTEENTH STREET, ARLINGTON, VA, 22209

(21) Appl. No.: 09/865,281

(22) Filed: May 29, 2001

Related U.S. Application Data
(63) Continuation-in-part of application No. 09/070,907, filed on May 4, 1998, now Pat. No. 6,238,667.

(51) Int. Cl. A61K 39/395; C07H 21/04; C12P 21/02; C12N 5/06

(52) U.S. Cl. 424/178.1; 435/69.1; 435/320.1; 435/326; 530/391.1; 536/23.53

(57) ABSTRACT

A fusion protein is made up of (1) an antibody and (2) a peptide having a biological activity selected from the group consisting of immuno-stimulatory, membrane transport and homophilic activities wherein the peptide is connected to the antibody at a site that does not interfere with antigen binding of the antibody. The fusion protein is made by the steps of creating a fusion gene including a nucleic acid sequence encoding an antibody and a nucleic acid sequence encoding the peptide, wherein the nucleic acid sequence encoding the peptide is located inside the nucleic acid sequence encoding the antibody at a site wherein, when the fusion is expressed, the fusion protein created thereby comprises the antibody and the peptide, wherein the peptide is connected to the antibody at a site that does not interfere with antigen binding of the antibody, and expressing the fusion gene to create the fusion protein. N-terminal residues or the C-terminal residues of H or L chains. The expression of such fused genes is typically done in mammalian cell lines as so-called fusion protein.

### CROSS REFERENCE TO RELATED APPLICATIONS

The present application is a continuation-in-part of U.S. patent application Ser. No. 09/070,907, filed May 4, 1998, the disclosure of which is incorporated herein by reference.

### FIELD OF THE INVENTION

The present invention relates to fusion proteins of biologically active peptides and antibodies. Specifically, the fusion proteins of the present invention combine the molecular recognition of antibodies with a biological activity such as immunostimulatory activity, membrane transport activity, and homophilic activity.

### BACKGROUND OF THE INVENTION

Antibodies have been praised as “magic bullets” to combat disease. However, the promises made for antibodies were never fully realized. This is in part due the fact that antibodies represent only one arm of the immune defense, where T-cells provide the other strategy in immune defense. However, antibodies are ideal targeting and delivery devices. They are adapted for long survival in blood, have sites which help vascular and tissue penetration and are functionally linked with a number of defense mechanisms of the innate immunity. One such mechanism is the complement system which helps to destroy pathogens and is involved in the regulation of immune responses. For example the complement fragment C3d binds to the CR2 receptor on B-cells, which is also the binding site for Epstein-Barr virus. Binding of Epstein-Barr virus to CR2 activates B-cells. Accumulated evidence has shown that the CR2 receptor (CD19/Cd20/CD81 complex) has an immuno-stimulatory role and is activated by C3d.

Another example of how antibodies can be used to enhance the immune response has been demonstrated by the work of Zanetti and Bona (Zanetti, M. Nature 355: 466-477, 1992; Zaghouani H.; Anderson S. A., Sperbeer K. E., Daian C. Kennedy R. C., Mayer L. and Bona C. A. 1995 Proc. Nat. Acad. Science U.S. 92: 631-635). These authors have replaced the CDR3 sequence of the Ig heavy chain with a sequence resembling T-cell and B-cell antigens (epitopes) using molecular biology methods and have shown that these modified antibodies induce potent immune response specific for the inserted groups.

The biological properties of the antibodies can be enhanced with respect to overall avidity for antigen and the ability to penetrate cellular and nuclear membranes. Antigen binding is enhanced by increasing the valency of antibodies such as in pentameric IgM antibodies. Valency and avidity is also increased in certain antibodies which are self binding or homophilic (Kang, C. Y., Cheng, H. L., Rudikoff, S. and Kohler, H. J. Exp. Med. 165:1332, 1987). Xiyun, A. N., Evans, S. V., Kaminki, M. J., Fillies, S. F. D., Resifeld, R. A., Noughton, A. N. and Chapman, P. B. J. Immunol. 157: 1582-1588 (1996)). A peptide in the heavy chain variable region was identified which inhibited self-binding (Kang, C. Y. Brunck, T. K., Kieber-Emmons, T., Blalock, J. E. and Kohler, H., Science, 240: 1034-1036, 1988). The insertion of self-binding peptide sequence into an antibody endows the property of self-binding and increases the valency and overall avidity for the antigen.

Similarly the addition of a signal peptide to antibodies facilitates transmembrane transport as demonstrate by Rojas et al, Nature Biotechnology, 16: 370-375 (1998). Rojas et al. have generated a fusion protein which contained a 12 mer peptide and have shown that this protein has cell membrane permeability.

Antibodies have been used as delivery devices for several biologically active molecules, such as toxins, drugs and cytokines. Often fragments of antibodies, Fab or scFv, are preferred because of better tissue penetration and reduced “stickiness”.

There are two practical methods for attaching molecules such as peptides to antibody molecules. One method is to use chemical crosslinking, such as the affinity-crosslinking method described in co-pending application Ser. No. 09/070,907. Another method is to design a fusion gene containing DNA encoding the antibody and the peptide and to express the fusion gene, which method is the subject of the present application.

Antibody fusion proteins have typically been engineered with entire genes of large proteins or domains of such proteins that produce a biological function. Small peptide-antibody fusion proteins are typically made to facilitate the purification or characterization of the antibody.

Methods of creating fusion proteins are described, for example, in the following U.S. patents, incorporated herein by reference: U.S. Pat. No. 5,563,046 to Mascarenhas et al; U.S. Pat. No. 5,645,835 to Fell, Jr.; U.S. Pat. No. 5,668,225 to Murphy; U.S. Pat. No. 5,698,679 to Nemazee; U.S. Pat. No. 5,763,733 to Whitlow et al; U.S. Pat. No. 5,811,265 to Quertermous et al; U.S. Pat. No. 5,908,626 to Chang et al; U.S. Pat. No. 5,969,109 to Bona et al; U.S. Pat. No. 6,008,319 to Epstein et al; U.S. Pat. No. 6,117,656 to Seed; U.S. Pat. No. 6,121,424 to Whitlow et al; U.S. Pat. No. 6,132,992 to Ledbetter et al; U.S. Pat. No. 6,207,804 to Huston et al; and U.S. Pat. No. 6,224,870 to Segal. Methods of creating Ig fusion proteins are described, for example, in Antibody Engineering, 2nd Edition. ed.: Carl A. K. Borrebaeck, Oxford University Press 1995, and in “Molecular Cloning: A Laboratory Manual, Second Ed., Cold Spring Harbor Press, 1989, incorporated herein by reference.

### SUMMARY OF THE INVENTION

The present invention provides a fusion protein comprising an antibody and a peptide having a biological activity selected from the group consisting of immuno-stimulatory, membrane transport and homophilic activities, wherein the peptide is located at a site in the antibody so that the incorporated peptide does not compromise the antigen recognition of the antibody. In the present invention, this is accomplished by a method comprising the steps of creating a fusion gene comprising a nucleic acid sequence encoding an antibody and a nucleic acid sequence encoding the peptide, wherein the nucleic acid sequence encoding the peptide is located inside the nucleic acid sequence encoding the antibody at a site wherein, when the fusion is expressed, the fusion protein that is created thereby includes the antibody plus the peptide, and the peptide is connected to the antibody at a site that does not interfere with antigen binding of the antibody, and expressing the fusion gene to create the fusion protein. In particular, the fusion protein may be created by providing a gene encoding an antibody, wherein the gene is mutated to contain a restriction site, wherein the restriction site is located away from any section of the gene that encodes an antigen-binding site of the antibody, inserting a DNA sequence encoding a peptide having a biological activity selected from the group consisting of immuno-stimulatory, membrane transport and homophilic activities into restriction site of the gene encoding the antibody to create a fusion gene, and wherein the DNA sequence encoding the peptide is inserted so that it is in-frame with the gene encoding the antibody, and expressing the fusion gene to create a fusion protein.

The invention also provides a composition and a pharmaceutical composition comprising a fusion protein of a peptide having a biological activity selected from the group consisting of immuno-stimulatory, membrane transport and homophilic activities and an antibody.

The invention of creating fusion proteins of biologically active peptides and antibodies includes peptides which comprise self-binding, stimulate lymphocytes and allow transport across biological membranes.

The above and other objects of the invention will become readily apparent to those of skill in the relevant art from the following detailed description and figures, wherein only the preferred embodiments of the invention are shown and described, simply by way of illustration of the best mode of carrying out the invention. As is readily recognized the invention is capable of modifications within the skill of the relevant art without departing from the spirit and scope of the invention.

### DETAILED DESCRIPTION OF THE INVENTION

The present invention describes a method for creating fusion proteins of an antibody and a peptide having a biological activity selected from the group consisting of immuno-stimulatory, membrane transport and homophilic activities.

In particular, the present invention provides a fusion protein comprising an antibody and a peptide having a biological activity selected from the group consisting of immuno-stimulatory, membrane transport and homophilic activities, wherein the peptide is located at a site in the antibody so that the incorporated peptide does not compromise the antigen recognition of the antibody. In the present invention, this is accomplished by a method comprising the steps of creating a fusion gene comprising a nucleic acid sequence encoding an antibody and a nucleic acid sequence encoding the peptide, wherein the nucleic acid sequence encoding the peptide is located inside the nucleic acid sequence encoding the antibody at a site wherein, when the fusion is expressed, the fusion protein that is created thereby includes the antibody plus the peptide, and the peptide is connected to the antibody at a site that does not interfere with antigen binding of the antibody, and expressing the fusion gene to create the fusion protein. In particular, the fusion protein may be created by providing a gene encoding an antibody, wherein the gene is mutated to contain a restriction site, wherein the restriction site is located away from any section of the gene that encodes an antigen-binding site of the antibody, inserting a DNA sequence encoding a peptide having a biological activity selected from the group consisting of immuno-stimulatory, membrane transport and homophilic activities into restriction site of the gene encoding the antibody to create a fusion gene, and wherein the DNA sequence encoding the peptide is inserted so that it is in-frame with the gene encoding the antibody, and expressing the fusion gene to create a fusion protein.

The Expression of Ig-fusion Proteins (Adapted From Antibody Engineering, 2nd Edition. ed.: Carl A. K. Borrebaeck, Oxford University Press 1995)

Ig fusion proteins have the advantage of joining the antibody combining specificity and/or antibody effector functions with molecules contributing unique properties. The ability to produce this family of proteins was first demonstrated when c-myc was substituted for the Fc of the antibody molecule,(Neuberger M S, Williams G T and Fox R O. Nature 125:604, 1984) but many examples now exist. Ab fusion proteins can be achieved in several different ways. In one approach non-Ig sequences are substituted for the variable region; the molecule replacing the V region provides specificity of targeting with the antibody contributing properties such as effector functions and improved pharmacokinetics. Examples include IL-2 and CD4. Alternatively, non-Ig sequences can be substituted for or attached to the constant region. The resulting molecules retain the binding specificity of the original antibody but gain characteristics from the attached protein. Depending on the position of the substitution, different antibody-related effector functions and biologic properties will be retained.

Vectors for the Construction of IgG Fusion Proteins

A series of vectors has now been produced that permits the fusion of proteins at different positions within an antibody molecule, thereby facilitating the construction of fusion proteins with different properties. Using these vectors it is possible to produce a family of fusion proteins with molecules of differing molecular weight, valence, and having different subsets of the functional properties of the antibody molecule.

As a specific example of how to facilitate the construction of fused genes, site-directed mutagenesis was used to generate unique restriction enzyme sites in the human IgG3 heavy chain gene. In this particular example, restriction sites were generated at the 3′ end of the CH1 exon, immediately after the hinge at the 5′ end of the CH2 exon, and at the 3′ end of the CH3 exon. The restriction sites thus produced were SnaB I at the end of CH1 by replacing TtgGTg with TacGTa, Pvu II at the beginning of CH2 by replacing CAcCTG with CAgCTG, and Ssp I at the end of CH3 replacing AATgag with AATatt. These manipulations provided a unique blunt-end cloning site at these positions. In all cases the restriction site was positioned so that after cleavage the Ig would contribute the first base of the codon. Human IgG3 with an extended hinge region of 62 amino acids was chosen for use as the immunoglobulin; when present this hinge should provide spacing and flexibility, thereby facilitating simultaneous antigen and receptor binding. An EcoR I site was also introduced at 3′ of the IgG3 gene to provide a 3′ cloning site and polyA addition signal. Although initially designed for use with growth factors, these restrictions sites can be used to position any novel sequence at defined positions in the antibody. Also, using these cloning cassettes the variable region can easily be changed. Similar techniques may be used to generate suitable restriction sites in other antibody genes.

Production of A Fusion Gene

As a first step in the production of a fusion protein, a blunt-end restriction site must be introduced at the desired position into the 5′ end of the gene to be fused. In order to maintain the correct reading frame, the site must be positioned so that after cleavage it will contribute two bases to the codon. If the objective is to make a fusion protein with the complete molecule, the restriction site is usually introduced at the position of any post-translational processing, such as after the leader sequence. Alternatively, if the objective is to use only a portion of the protein, the blunt-end site can be introduced at any position within the gene, but attention must always be paid to maintaining the correct reading frame. Additionally, if there is carboxylterminal post-translational processing of the fused protein, it is frequently desirable to introduce a stop-codon at this processing site.

A major concern when producing fusion proteins is maintaining the biologic activities of all of the components. The production of fusion proteins with antibodies is facilitated by the domain structure of the antibody, and all of the cloning sites have been positioned immediately following an intact domain. In this configuration the correct folding of the immunoglobulin should be assured. The folding of the attached protein depends on its structure and where it is fused. Whenever structural information is available, it is desirable to produce the fusion at a position that will maintain the structural integrity of the attached protein.

To produce quantities of protein sufficient for functional analysis, it is desirable to have the protein secreted into the medium. While in the examples reported to date, assembled fusion proteins have been assembled and secreted, this remains a concern when designing additional fusion proteins.

The method to design a fusion gene that contains a biologically activity peptide as part of the heavy or light chain gene can use established antibody engineering protocols (Antibody Engineering, 2nd Edition. ed.: Carl A. K. Borrebaeck, Oxford University Press 1995. Chapter 9, pages 267-293). The peptide can fused either to N-terminal residues or the C-terminal residues of H or L chains. The expression of such fused genes is typically done in mammalian cell lines, although other expression systems, such as, for example, bacteria or yeast expression systems, may be used.

The peptide of the invention has a biological activity selected from the group consisting of immuno-stimulatory, membrane transport and homophilic activities. Examples include immuno-stimulatory or immuno-regulatory activity. The peptide may, for example, be a hormone, ligand for cytokines or a binding site derived from natural ligands for cellular receptors. In a preferred embodiment the peptide is derived from C3d region 1217-1232 and ranges from about 10 to about 16 mer. In an alternative embodiment the peptide is a 16 mer peptide derived from the C3d region 1217-1232.

The peptide may be bound to an antibody which is a full-length immunoglobulin molecule or a variable domain fragment of an antibody. As used herein, the term “antibody” refers generally to a heavy or light chain immunoglobulin molecule or any function combination or fragment thereof containing an antigen binding site. The antibody is preferably specific for a cellular receptor, on a membrane structure such as a protein, glycoprotein, polysaccharide or carbohydrate, and on a normal cell or on tumor cells.

The use of peptides derived from the ligand site of C3d as an immunostimulatory component incorporated into antibodies has an unexpected utility as a molecular adjuvant. C3d has been used as molecular adjuvant as part of a complete fusion protein with hen egg lysozyme (HEL) by D. Fearon, et al., (Dempsey, P. W., Allison, M. E. D., Akkaraju, S., Goodnow, C. C. and Fearon, D. T., Science, 271:348, 1996). These authors have shown that a HEL- C3d fusion protein is up to 10,000 fold more immunogenic than free HEL (see International Patent Publication, WO96/17625).

Similar increases in immunogenicity have been observed with chemical cross-linked idiotype vaccines using a peptide derived from the C3d fragment in our recent animal studies (see examples below). It is believed that attaching C3d peptides to idiotype and anti-idiotype vaccines enhances the immunogenicity of these vaccines and substitutes for the need of attaching carrier molecules such as KLH in combination with strong adjuvants such Freund adjuvant which is not permitted by the FDA for humans.

In an alternative embodiment, the peptide may be derived from a human or non-human C3d region homologous to the human C3d residues at position 1217-1232 and ranges from about 10 to about 16 mer. Other applications of affinity cross-linking biologically active peptides to antibody vaccines include active peptides derived from cytokines. For example, a nonapeptide from the IL1-beta cytokine has been described (Antoni, et al., J. Immunol, 137:3201-04, 1986) which has immunostimulatory properties without inducing undesired side effects. Other examples of active peptides which can be inserted into antibodies in accordance with the invention include signal peptides, and peptides from the selfbinding locus of antibodies.

A variety of peptides are known having biological activities as hormones, ligands for cytokines or binding sites derived from natural ligands for cellular receptors.

### EXAMPLES

The following examples 1-3, while relating to C3d/antibody complexes that are created by affinity cross-linking, are provided to show the effects on the immune response provided by C3d peptides linked to antibodies.

### Example 1

Enhancement of an Anti-idiotype Vaccine.

3H1 is a murine anti-idiotype antibody (Bhattacharya-Chatterjee, et al., J. Immunol., 145:2758-65, 1990) which mimics the carcino-embryonic antigen (CEA). 3H1 induces in animals anti-CEA antibodies when used as KLH-conjugated vaccine in complete Freund's adjuvant. 3H1 has also been tested in a clinical phase I study where it induces antibodies which bind to CEA in approximately half of treated cancer patients. However no clinical response was observed in this study (Foon, et al., J. Clin. Invst., 96:334-342, 1995) due, in part, to low immunogenicity.

3H1 mAb was affinity cross-linked with a 13mer peptide (SEQ ID NO.:1) derived from the C3d region 1217-1232. The amino acid sequence was derived from of the Cd3 peptide and has the following sequence (SEQ ID NO. 1):KNRWEDPGKQLYNVEA-. BALB/c mice were immunized twice with 25 μg of C3d-3H1 in phosphate-saline solution intramuscular. 7 days after the last immunization mice were bled and sera were tested for binding to 8019 (Ab1 idiotype) and to the CEA expressing tumor line LS174T. As determined by FACS, sera from C3d-3H1 immune mice bind to LS174T tumor cells, while a control serum (normal mouse serum) showed only background fluorescence. Sera from mice immunized with C3d-3H1 were used in FACS of LS174T cells in a sandwich assay developed with FITC conjugated goat anti-mouse IgG. Control was a normal mouse serum. Cell number analyzed were plotted against relative fluorescence intensity on log 10 scale.

### Example 2

Furthermore, sera from mice immunized three times with either 3H1 (25 microgram in saline) or 3H1-C3d-peptide (affinity cross-linked, 25 microgram in saline) were also tested for Ab3 response. Mice were bled and sera were tested for binding to F(ab) of 3H1 in ELISA. Upon determining the binding of dilutions of mouse sera to 3H1 F(ab), it was found that while naked 3H1 does not induce Ab3 antibodies, 3H1-peptide does showing that the affinity-cross-linked 3H1 enhanced immunogenicity.

Other C3d peptides which may be used in the practice of the present invention include those reviewed in Lambris et al, “Phylogeny of the third component of complement, C3” in Erfi, A ed. New Aspects of Complement structure and function, Austin, R. D. Landes Co., 1994 p. 15-34, incorporated herein by reference in its entirety.

### Example 3

Enhancement of an Mouse Tumor Idiotype Vaccine (38C13).

38C13 is the idiotype expressed by the 38C13 B-lymphoma tumor cell line. The Levy group has developed this idiotype tumor vaccine model and has shown that pre-immunization with KLH-conjugated 38C13 Id can protect against challenge with 38C13 tumor cells in mice (Kaminski, M. S., Kitamura, K., Maloney, D. G. and Levy, R., J. Immunol, 138:1289, 1987). Levy and colleagues (Tao, M-H. and Levy, R., Nature 362:755-758, 1993) have also reported on the induction of tumor protection using a fusion protein (CSF-38C13), generated from a chimeric gene and expressed in mammalian cell culture fermentation. 38C13 Id proteins was affinity cross-linked with a 16 mer azido-peptide derived from the C3D region 1217-1232.

10 mice were immunized with 50 ug of C3d-38C13 conjugate in phosphate-saline solution intraperitoneally three times. After the third vaccination mice were challenge with 38C13 tumor cells. Control groups included mice vaccinated with 38C13-KLH in QS-21 (adjuvant), considered the “gold standard” in this tumor model, and mice injected with QS-21 alone. 7 out 10 mice vaccinated with the C3d-38C13 conjugate survived by day 35 after tumor challenge, as did mice vaccinated with the KLH-38C13 in QS-21. All control mice injected only with QS-21 had died by day 22.

C3H mice were immunized three times with either 38C13-KLH in QS-21 or with 38Cl3-C3d peptide without QS-21 (50 ug i.p.) Control mice were only injected with QS-21. Immunized and control mice were than challenged with 38C13 tumor cells and survival was monitored.

Results described in Examples 1-3 show that affinity-cross-linking of an immuno-stimulatory peptide to tumor anti-idiotype and idiotype vaccine antibodies can significantly enhance the immune response to the tumor and protect against tumor challenge. The vaccination protocol with the C3d-cross-linked vaccine did not include any adjuvant, such as Freund's adjuvant, or KLH conjugation, both of which are not permissible by the FDA for human use.

Some of the procedures used in the above examples are known; The active binding peptide of C3d (complement fragment) has been described by Lambris, et al., (PNAS, 82:4235-39, 1985) and is incorporated herein by reference in its entirety.

The following additional examples are provided to demonstrate the general technique of creating fusion proteins and to illustrate particular peptide having a biological activity selected from the group consisting of immuno-stimulatory, membrane transport and homophilic activities.

### Example 4

Fusion non-Ig Protein containing a Membrane Transferring Peptide (MTS-peptide) taken from Rojas, M, Donahue, J P, Tan, T. and Lin, Y-Z. Nature Biotechnology, 16: 370, 1998. Construction of the glutathion S-transferase-MTS peptide (GST-MTS) expression plasmids.

Two different GST-MTS expression plasmids were constructed so that, depending on the biological application, a target protein or protein domain could be produced with the hydrophobic MTS as either an amino-terminal or a carboxyl-terminal extension. For the construction of plasmids pGEX-3X-MTS I and pGEX3X-MTS2, the following complementary oligonucleotides were synthesized:

(SEQ ID NO. 2)

MTSI GATCGCAGCCGTTCTTCTCCCTGTTCTTCTTGCCGCACCCGGCGT
CGGCAAGAAGAGGGACAAGAAGAACGGCGTGGGCCCTAG

(SEQ ID NO. 3)

MTS2 GATCCCCGCAGCCGTTCTTCTCCCTGTTCTTCTTGCCGCACCCTA
GCGGGCGTCGGCAAGAAGAGGGACAAGAAGAACGGCGTGGGATTCGCTAG

After annealing, the double-stranded MTS I and MTS2 oligonucleotides were ligated in BamHI digested pGEX-3X (Smith, D. B. and Johnson, K. S. 1988. Single-step purification of polypeptides expressed in Escherichia coli as fusions with glutathione S-transferase. Gene 67:31). DNA sequence analysis confirmed that in each plasmid the MTS coding sequence was correct and in-frame with the GST coding sequence.

Construction of GST-Grb2SH2, GST-Grb2SH2-MTS, and GST-StatlSH2-MTS Expression Plasmids.

A DNA fragment encoding the human Grb2 SH2 domain (amino acid residues 54-164)(Lowenstein, E. J., Daly, R. J., Batzer, A. G., U, W., Margolis, B., Lammers, R et al 1992. The SH2 and SH3 domain-containing protein Grb2 links receptor tyrosine kinases to ras signaling. Cell 70:431) or the human Statl SH2 domain (residues 567-716) (Schindler, C., Fu, X.-Y, Impnota, T., Aebersold, R., and Darnell, J. E. Jr. 1992. Q. Proc. Natl Acad. Sci USA 89:7836) was synthesized from a Grb2 cDNA clone or a Statl cDNA clone by PCR. The primers used for PCR, each containing BamHI sites at their 5′ends, were as follows:

| | |
|---|---|
| Grb2 SH2: | (SEQ ID NO. 4) |

5′-CCGGATCCCCGAAATGAAACCACATCCGTGGTTTTTTGGC

(SEQ ID NO. 5)

and 5′-CCGGATCCCGAGGGCCTGGACGTATGTCGGCTGCTGTGG.

| | |
|---|---|
| Statl SH2: | (SEQ ID NO. 6) |

5′-CCGGATCCCCAAACACCTGCTCCCTCTCTGGAATGATGGG

(SEQ ID NO. 7)

and 5′-CCGGATCC-CTCTAGAGGGTGAACTTCAGACACAGAAAT.

The PCR products were digested with BamHI and ligated in BamHI-digested pGEX-3X or pGEX-3XMTS2. DNA sequence analysis of the vector/insert junctions confirmed that the GST-Grb2SH2, GST-Grb2SH2-MTS, and GST-StatlSH2-MTS translational reading frames were maintained in each expression plasmid.

Expression of MTS Fusion Protein

Expression and purification of GST fusion proteins. E. coli strain DHSor containing the appropriate expression plasmid 74 as grown in LB broth containing 100 μg/ml ampicillin at 37° C. GST fusion protein expression was induced by the addition of isopropyl, B-D-thiogalactoside (0.5 mM final concentration), and incubation at 37° C. was continued for 2-3 hours. GST fusion proteins were purified from bacterial cell lysates by glutathione-agarose affinity chromatography. (Smith, D. B. and Johnson, K. S. 1988. Gene 67:31) except that after sonication, cell lysates were cleared by centrifugation at 2000×g for 5 minutes prior to mixing with glutathione-agarose beads. Protein preparations were concentrated by ultrafiltration using a PMIO membrane (Amicon, Beverly, Mass.) and stored at 4° C. for immediate use or −70° C. for prolonged storage. Protein concentrations were determined spectrophotometrically at 280 nm. Immediately prior to their use in biological assays, protein concentrations were verified by SDS-PAGE using Coomassie brilliant blue staining intensity compared with wild-type GST of known concentration. To confirm the amino acid content of the MTS in GST-MTS proteins, the MTS peptide was cleaved from glutathione-agarose bound GST-MTSI with protease factor Xa essentially as described (Smith, D. B. and Johnson, K. S. 1988. Gene 67:31). The released MTS-containing peptide was purified by C, reverse-phase HPLC and characterized by mass spectrometry analysis as described (Smith, D. B. and Johnson, K. S. 1988. Gene 67:31). The released MTS-containing peptide was purified by C₁₈ reverse-phase HPLC and characterized by mass spectrometry as described (Lin, Y-Z., Yao, S., Veach, R. A., Torgerson, T. R., and Hawiger, J. 1995. J BioL Chem. 270:14255).

### Example 5

C3d -HEL fusion protein (taken from Dempsey et al. Science, 271: 348, 1996)

The complimentary DNA encoding HEL, C3d (H. Domdey et al Pro. Natl Acad Sci USA 79: 7619, 1982) doq pre-pro-insulin signal sequence (M. E. Taylor and K. Drickamer, Biochem. J. 274, 575 (1991), and the (G4S)₂ linker were amplified by polymerase chain reaction. The epitope tag and stop codon were coded for by oligonucleotide linkers. Fusion protein cassetes were assembled in tandem: doq pre-pro-insulin signal sequence, HEL, and one to three copies of C3d linked by (G₄S)₂ in pSG5 (Stratagene). The HEL-C3d3 cassette was subcloned into the A71d vector. The plasmids pSG.HEL, pSG.HEL.C3d, and pSG.HEL.C3d2 were co-transfected with pSV2-neo into L cells and A71d. HEL.C3d3 was transiently expressed in COS cells. Recombinant proteins were punfied by affnity chromatography on YL ½ antibody (H. Skinner et al., J. BioL Chem. 266, 14163 1991) and fractionation on Sephacryl S-200 (Pharmacia).

Fusion tails are useful at the lab scale and have potential for enhancing recovery using economical recovery methods that are easily scaled up for industrial downstream processing. Fusion tails can be used to promote secretion of target proteins and can also provide useful assay tags based on enzymatic activity or antibody binding. Many fusion tails do not interfere with the biological activity of the target protein and in some cases have been shown to stabilize it. Nevertheless, for the purification of authentic proteins a site for specific cleavage is often included, allowing removal of the tail after recovery.

Fusion Tails for the Recovery and Purification of Recombinant Proteins. [Adapted From Ford C F, Suominen I, Glatz C E (Protein Expr. Purif 2-3: 95-107, 1991]

The fusion protein of the present invention may also include a fusion tail such as has been developed to promote efficient recovery and purification of recombinant proteins from crude cell extracts or culture media. In these systems, a target protein is genetically engineered to contain a C- or N-terminal polypeptide tail, which provides the biochemical basis for specificity in recovery and purification. Tails with a variety of characteristics have been used:

(1) entire enzymes with affinity for immobilized substrates or inhibitors;

(2) peptide-binding proteins with affinity to immunoglobulin G or albumin;

(3) carbohydrate-binding proteins or domains;

(4) a biotin-binding domain for in vivo biotination promoting affinity of the fusion protein to avidin or streptavidin;

(5) antigenic epitopes with affinity to immobilized monoclonal antibodies;

(6) poly(His) residues for recovery by immobilized metal affinity chromatography; and

(7) other poly(amino acid)s, with binding specificity based on properties of the amino acid side chain.

Fusion tails are useful at the lab scale and have potential for enhancing recovery using economical recovery methods that are easily scaled up for industrial downstream processing. Fusion tails can be used to promote secretion of target proteins and can also provide useful assay tags based on enzymatic activity or antibody binding. Many fusion tails do not interfere with the biological activity of the target protein and in some cases have been shown to stabilize it. Nevertheless, for the purification of authentic proteins, a site for specific cleavage is often included, allowing removal of the tail after recovery.

The present invention describes the generation of an antibody-peptide fusion protein that enhances the biological and immunological activity of the antibody without changing the antibody specificity for the corresponding antigen. In the genetically engineered fusion protein mimics the chemically engineered chimeric antibodies described in patent application Ser. No. 09/070,907. Specifically, the present invention provides the generation of antibody fusion proteins containing the complete or partial autophilic 24 mer peptide, the membrane transport peptide (MTS) or the C3d peptide, all described above.

The invention also provides a composition and a pharmaceutical composition comprising a fusion protein made up of (1) an antibody and (2) a peptide having a biological activity selected from the group consisting of immuno-stimulatory, membrane transport and homophilic activities wherein the peptide is connected by peptide bonds to the antibody at a site that does not interfere with antigen binding of the antibody.

Any antibody may be used in the peptide/antibody complex of the invention. Preferred antibodies are anti-idiotype antibodies. For example, anti-idiotype antibody 3H1 may be used (see “Anti-idiotype Antibody Vaccine (3H1) that Mimics the Carcinoembryonic Antigen (CEA) as an Adjuvant Treatment”, Foon, et al., Cancer Weekly, Jun. 24, 1996). Other anti-idiotype antibodies which may be used in the present invention include, for example, anti-idiotype antibody to chlamydia glycolipid exoantigen (U.S. Pat. No. 5,656,271; anti-idiotype antibody 1A7 for the treatment of melanoma and small cell carcinoma (U.S. Pat. No. 5,612,030); anti-idiotype antibody MK2-23 anti-melanoma antibody (U.S. Pat. No. 5,493,009); anti-idiotypic gonococcal antibody (U.S. Pat. No. 5,476,784) Pseudomonas aeruginosa anti-idiotype antibody (U.S. Pat. No. 5,233,024); antibody against surface Ig of Human B cell tumor (U.S. Pat. No. 4,513,088); and monoclonal antibody BR96 (U.S. Pat. No. 5,491,088). Any restrictions on peptide length are those practical limitations associated with peptide synthesis and not restrictions associated with practice of the method of the invention.

Additionally, self-binding peptides such as those disclosed in (Kang, C. Y. Brunck, T. K., Kiever-Emmons, T., Blalick, J. E. and Kohler, H., “Inhibition of self-binding proteins (auto-antibodies) by a VH-derived peptide, Science, 240: 1034-1036, 1988, incorporated herein by reference in its entirety) used in the method of the present invention.

Additionally, signal peptides such as those disclosed in Roias, et al., “Genetic Engineering of proteins with cell membrane permeability”, Nature Biotechnology, 16: 370-375 (1988) and Calbiochem Signal Transduction Catalogue 1997/98, incorporated herein by reference in their entireties, may be used in the method of the invention.

The peptide may be designed to have inverse hydropathic character and exhibits mutual affinity and homophilic (self) binding within the peptide, in accordance with the disclosure of U.S. Pat. No. 5,523,208 (incorporated herein by reference in its entirety).

The compositions of the invention are useful in pharmaceutical compositions for systemic administration to humans and animals in unit dosage forms, sterile solutions or suspensions, sterile non-parenteral solutions or suspensions oral solutions or suspensions, oil in water or water in oil emulsions and the like, containing suitable quantities of an active ingredient. Topical application can be in the form of ointments, creams, lotions, jellies, sprays, douches, and the like. The compositions are useful in pharmaceutical compositions (wt %) of the active ingredient with a carrier or vehicle in the composition in about 1 to 20% and preferably about 5 to 15%.

The above parenteral solutions or suspensions may be administered transdermally and, if desired a more concentrated slow release form may be administered. The cross-linked peptides of the invention may be administered intravenously, intramuscularly, intraperitoneally or topically. Accordingly, incorporation of the active compounds in a slow release matrix may be implemented for administering transdermally. The pharmaceutical carriers acceptable for the purpose of this invention are the art known carriers that do not adversely affect the drug, the host, or the material comprising the drug delivery device. The carrier may also contain adjuvants such as preserving stabilizing, wetting, emulsifying agents and the like together with the penetration enhancer of this invention. The effective dosage for mammals may vary due to such factors as age, weight activity level or condition of the subject being treated. Typically, an effective dosage of a compound according to the present invention is about 10 to 500 mg, preferably 2-15 mg, when administered by suspension at least once daily. Administration may be repeated at suitable intervals.

The purpose of the above description and examples is to illustrate some embodiments of the present invention without implying any limitation. It will be apparent to those of skill in the art that various modifications and variations may be made to the composition and method of the present invention without departing from the spirit or scope of the invention. All patents and publications cited herein are incorporated by reference in their entireties.

What is claimed is:

1. A method of creating a fusion protein made up of (1) an antibody and (2) a peptide having a biological activity selected from the group consisting of immuno-stimulatory, membrane transport and homophilic activities wherein the peptide is connected to the antibody at a site that does not interfere with antigen binding of the antibody, the method comprising the steps of
creating a fusion gene comprising a nucleic acid sequence encoding an antibody and a nucleic acid sequence encoding the peptide, wherein the nucleic acid sequence encoding the peptide is located inside the nucleic acid sequence encoding the antibody at a site wherein, when the fusion is expressed, the fusion protein created thereby comprises the antibody and the peptide, wherein the peptide is connected to the antibody at a site that does not interfere with antigen binding of the antibody, and
expressing the fusion gene to create the fusion protein.

2. The method of claim 1 wherein the antibody is an IgG heavy chain or light chain.

3. The method of claim 1 wherein the antibody is an immunoglobulin fragment containing an antigen binding site.

4. A method of creating a fusion protein made up of (1) an antibody and (2) a peptide having a biological activity selected from the group consisting of immuno-stimulatory, membrane transport and homophilic activities wherein the peptide is connected to the antibody at a site that does not interfere with antigen binding of the antibody, the method comprising the steps of
providing a gene comprising a nucleic acid sequence encoding an antibody, wherein the gene is mutated to contain a restriction site, wherein the restriction site is located away from any section of the gene that encodes an antigen-binding site of the antibody,
creating a fusion gene by inserting a nucleic acid sequence encoding a peptide having a biological activity selected from the group consisting of immuno-stimulatory, membrane transport and homophilic activities into the restriction site of the gene comprising the nucleic acid sequence encoding the antibody, and wherein the nucleic acid sequence encoding the peptide is inserted so that it is in-frame with the nucleic acid sequence encoding the antibody or antibody fragment, and
expressing the fusion gene to create a fusion protein.

5. The method of claim 4 wherein the antibody is a heavy or light chain of human IgG, IgA or IgM.

6. The method of claim 4 wherein the antibody is a heavy chain of human IgG and the restriction site is located at the 3′ end of the CH1 exon.

7. The method of claim 6 wherein the restriction site is created by locating a sequence of ttgqtg at the 3′end of the CH1 exon and replacing the sequence of ttggtg with a sequence of tacgta, thereby creating an SnaB I restriction site.

8. The method of claim 4 wherein the antibody is a heavy chain of human IgG and the restriction site is located after the hinge at the 5′ end of the CH2 exon.

9. The method of claim 8 wherein the restriction site is created by locating a sequence of cacctg immediately after the hinge at the 5′ end of the CH2 exon and replacing the sequence of cacctg with a sequence of cagctg, thereby creating an Pvu II restriction site.

10. The method of claim 4 wherein the antibody is a heavy chain of human IgG3 and the restriction site is located at the 3′ end of the CH3 exon.

11. The method of claim 10 wherein the restriction site is created by locating a sequence of aatgag at the 3′ end of the CH3 exon and replacing the sequence of aatgag with a sequence of aataat, thereby creating an Ssp I restriction site.

12. The method of claim 1, wherein said peptide is derived from a human or non-human C3d region homologous to the human C3d residues at position 1217-1232 and ranges from about 10 to about 16 mer.

13. The method of claim 1, wherein said peptide is a 16 mer peptide derived from a human or non-human C3d region homologous to the human C3d residues at position 1217-1232.

14. The method of claim 1, wherein said antibody is specific for a cellular receptor, or a membrane structure on a normal cell or on tumor cells.

15. The method of claim 1, wherein said peptide is selected from the group consisting of hormones, ligands for cytokines and binding sites derived from natural ligands for cellular receptors.

16. The method of claim 1, wherein said peptide has inverse hydropathic character and said peptide exhibits mutual affinity and homophilic binding, within the length of said peptide.

17. A method claim 1 wherein the antibody is a heavy or light chain immunoglobulin molecule and wherein the nucleic acid sequence encoding the peptide is located inside the nucleic acid sequence encoding the antibody at a site so that when the fusion gene is expressed, the peptide is attached directly to the C-terminal or the N-terminal of the heavy or light chain.

18. A fusion gene for expressing a fusion protein made up of (1) an antibody and (2) a peptide having a biological activity selected from the group consisting of immuno-stimulatory, membrane transport and homophilic activities wherein the peptide is connected to the antibody at a site that does not interfere with antigen binding of the antibody, the fusion gene comprising
a nucleic acid sequence encoding an antibody and
a nucleic acid sequence encoding a peptide having a biological activity selected from the group consisting of immuno-stimulatory, membrane transport and homophilic activities,
wherein the nucleic acid sequence encoding the peptide is located inside the nucleic acid sequence encoding the antibody at a site wherein, when the fusion is expressed, the fusion protein created thereby comprises the antibody and the peptide, wherein the peptide is connected to the antibody at a site that does not interfere with antigen binding of the antibody.

19. A fusion gene for expressing a fusion protein made up of (1) an antibody and (2) a peptide having a biological activity selected from the group consisting of immuno-stimulatory, membrane transport and homophilic activities wherein the peptide is connected to the antibody at a site that does not interfere with antigen binding of the antibody, wherein the fusion gene is made by a process comprising the steps of
providing a gene comprising a nucleic acid sequence encoding an antibody, the gene being mutated to contain a restriction site, wherein the restriction site is located away from any section of the gene that encodes an antigen-binding site of the antibody,
inserting a nucleic acid sequence encoding a peptide having a biological activity selected from the group consisting of immuno-stimulatory, membrane transport and homophilic activities into the restriction site of the gene, and wherein the nucleic acid sequence encoding the peptide is inserted so that it is in-frame with the nucleic acid sequence encoding the antibody or antibody fragment.

20. The fusion gene of claim 18 wherein the antibody encoded by the gene is a heavy or light chain immunoglobulin molecule and wherein the nucleic acid sequence encoding the peptide is located inside the nucleic acid sequence encoding the antibody at a site so that when the fusion gene is expressed, the peptide is attached directly to the C-terminal or the N-terminal of the heavy or light chain.

21. A fusion protein made up of (1) an antibody and (2) a peptide having a biological activity selected from the group consisting of immuno-stimulatory, membrane transport and homophilic activities wherein the peptide is connected by peptide bonds to the antibody at a site that does not interfere with antigen binding of the antibody.

22. The fusion protein of claim 21 wherein the antibody is a light chain or heavy chain immunoglobulin molecule and wherein the peptide is attached to the C-terminal or the N-terminal of the light chain or heavy chain.

23. A fusion protein made up of (1) an antibody and (2) a peptide having a biological activity selected from the group consisting of immuno-stimulatory, membrane transport and homophilic activities wherein the peptide is connected to the antibody at a site that does not interfere with antigen binding of the antibody, wherein the fusion protein is created by a process comprising the steps of
creating a fusion gene comprising a nucleic acid sequence encoding an antibody and a nucleic acid sequence encoding the peptide, wherein the nucleic acid sequence encoding the peptide is located inside the nucleic acid sequence encoding the antibody at a site wherein, when the fusion is expressed, the fusion protein created thereby comprises the antibody and the peptide, wherein the peptide is connected to the antibody at a site that does not interfere with antigen binding of the antibody, and
expressing the fusion gene to create the fusion protein.

24. The composition of claim 23, wherein said antibody is specific for a cellular receptor on a normal cell or on tumor cells.

25. The composition of claim 23, wherein said antibody is a full-length immunoglobulin molecule or a variable domain fragment of an antibody.

26. The composition of claim 23 wherein the peptide has inverse hydropathicity within the length of said peptide.


SEQUENCE LISTING (7 sequences)

SEQ ID NO: 1 - protein, 16 aa, Artificial
  PEPTIDE (1)..(16): Synthesized peptide with sequence derived from position 1217-1232
KNRWEDPGKQLYNVEA

SEQ ID NO: 2 - DNA, 84 bp, Artificial
  misc_feature (1)..(84): Synthesized oligonucleotide
gatcgcagccgttcttctccctgttcttcttgccgcacccggcgtcggcaagaagagggacaagaagaacggcgtgggccctag

SEQ ID NO: 3 - DNA, 95 bp, Artificial
  misc_feature (1)..(95): Synthesized oligonucleotide
gatccccgcagccgttcttctccctgttcttcttgccgcaccctagcgggcgtcggcaagaagagggacaagaagaacggcgtgggattcgctag

SEQ ID NO: 4 - DNA, 40 bp, Artificial
  misc_feature (1)..(40): Synthesized oligonucleotide primer for Grb2 SH2 cDNA
ccggatccccgaaatgaaaccacatccgtggttttttggc

SEQ ID NO: 5 - DNA, 39 bp, Artificial
  misc_feature (1)..(39): Synthesized oligonucleotide primer for Grb2 SH2 cDNA
ccggatcccgagggcctggacgtatgtcggctgctgtgg

SEQ ID NO: 6 - DNA, 40 bp, Artificial
  misc_feature (1)..(40): Synthesized oligonucleotide primer for Stat1 SH2 cDNA
ccggatccccaaacacctgctccctctctggaatgatggg

SEQ ID NO: 7 - DNA, 38 bp, Artificial
  misc_feature (1)..(38): Synthesized oligonucleotide primer for Stat1 SH2 cDNA
ccggatccctctagagggtgaacttcagacacagaaat